(19) United States
(12) Patent Application Publication
(10) Pub. No.: US 2012/0077184 A1
(43) Pub. Date: Mar. 29, 2012

(54) ELECTROMAGNETIC MULTIPLEX ASSAY BIOSENSOR

(75) Inventors: EMILY SEA HU, MENLO PARK, CA (US); Soon Pak Shinji Yue, Westford, MA (US)

(73) Assignee: STARKDX INCORPORATED, Union City, CA (US)

(21) Appl. No.: 12/892,641

(22) Filed: Sep. 28, 2010

(51) Int. Cl. G01N 33/53; C12M 1/34; C12Q 1/68; C12Q 1/02; C12Q 1/70

(52) U.S. Cl. 435/5; 435/29; 435/7.1; 435/287.2; 435/287.1; 435/6.1; 422/504

(57) ABSTRACT

A method is provided for determining the presence of multiple different target analytes in a liquid sample using electrophoretic separation and magnetic labels within a self-contained reaction cartridge and an external magnetic sensor for detection. Magnetic labels are bound to target analytes through specific binding elements. By electrophoretic separation, the multiple different targets can be sorted according to their specific sizes and inherent molecular charges for better detection resolution and specificity. After the separation process, the target analytes are then recognized and trapped by the detection binding elements within the reaction cartridge. A magnetic field generator provides a changeable magnetic field that causes the bounded magnetic labels and target analytes to produce a resonance disruption detectable by a magnetic sensor. The sensor can provide a digital binary value to indicate whether or not a label particle is bound and that determines the presence of target analytes.

### CROSS REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF INVENTION

1. Field of the Invention

The invention relates to the field of molecular biology and immunological testing with nanomaterials, more specifically to the field of cancer and point-of-care diagnostics. In particular the invention relates to methods of detecting multiple analytes from a single liquid sample using magnetic sensors and magnetic nanoparticles.

2. Background of the Invention

Medical doctors, scientists, and pharmaceutical companies need fast, easy-to-use biomolecule detection methods that generate unambiguous results to accurately detect and diagnose rapid progressive diseases such as cancers and infectious diseases. Speed of accurate detection can determine many life or death situations.

Traditional binding bioassays such as immunoassays (ELISA), DNA hybridization assays, and receptor-based assays are extensively used in diagnostic tests for a wide variety of analyte target molecules such as biomarkers, antibodies, viral and bacterial particles, deoxyribonucleic acid (DNA), ribonucleic acid (RNA), proteins, and biological receptors. Depending on the analyte, these tests typically take between 48 hours to three days at fairly high costs to produce moderate results that physicians can use to diagnose a disease state. Generally, users of such diagnostic tests need a high level of technical expertise to complete the bioassays using expensive equipment. The clinical interpretation is also very subjective and typically based on the detectable changes in the test reactions.

In a conventional ELISA bioassay, binding or capture molecules recognize and bind their specific target molecules in a sample, followed by the binding of the detection molecule usually the detection antibody. The detection molecule is typically labeled with a specific moiety that can generate a measurable signal thereby indicate the presence or absence of a target molecule. Radioactive, fluorescent, chemilummescent, magnetic and/or enzymatic labels have been used as such moiety to generate the signals. After through washings and removal of excess labeled molecules, the amount of bound label may be measured. This method is limited by its sensitivity, multiplexing capacity or, most importantly, uncontrollable response to the composition of complex biological samples. Detection across varied samples is crucial; for instance, a urologist may provide urine, a neurologist cerebrospinal fluid, a cardiologist blood or an oncologist cell lysates. The diversity of such matrices has hindered the generalizability and sensitivity of the majority of protein detection platforms, thus greatly reducing their clinical utility.

A large number of variations on the above-described binding assay methodologies have been described. For a complete description, see a laboratory handbook such as P. Tijssen, Practice and Theory of Enzyme Immunoassays, Elsevier Science Publishers, Amsterdam, 1985, the entire contents of which are incorporated herein by reference. The common feature of all binding assays is that labeled binding molecules adhere to a solid substrate in numbers that reflect the concentration of the target molecule.

Polymerase Chain Reaction (PCR) and the more recent real-time PCR, are commonly used methods for amplifying and detecting specific DNA or RNA sequences in a given sample. One of the main limitations of PCR assays is that cell integrity is lost during DNA/RNA extraction, thus preventing the analysis of cell morphology and phenotype. DNA polymerase are proficient enough to efficiently amplify DNA products up to a few thousand basepairs (2-5 kb). PCRs of longer products are less efficient due to enzyme activity loss, and inaccuracies introduced by longer PCRs. Adding fresh DNA polymerase helps with enzyme activity lost due to the half life of the polymerase, however this does not help when accurate PCR is required. PCR or real-time PCR requires a highly trained technician with expensive equipment to generate results.

Different variations on the PCR diagnostic methodologies have been described. For a complete description, see a laboratory handbook such as G. Viljoen, L. Nel, and J. Crowther, Molecular Diagnostic PCR Handbook, Springer, Netherlands, 2005, the entire contents of which are incorporated herein by reference. The common feature of all PCR assays is that DNA and/or RNA are extracted from cellular contents followed by enzymatic amplification of short DNA fragments (amplicons) through a repetitive heating and cooling process and the subsequent detection of amplicons.

Newer technologies such as nanowires (reviewed in P. Nair and M. Alam, Design Considerations of Silicon Nanowire Biosensors, IEEE Transaction on Electron Devices, 2007, 54:12, 3400-3408) microcantilevers (described in U.S. Pat. No. 5,719,324 issued Feb. 17, 1998 by T. G. Thundat and E. A Wachter describes an improved sensor attached to a transducer; G. H. Wu, et. al. Bioassay of prostate-specific antigen (PSA) using microcantilevers Nature Biotechnology, 2001, 19, 856-860), carbon nanotubes (described in P. F. Qi, et. al, Toward Large Arrays of Multiplex Functionalized Carbon Nanotube Sensors for Highly Sensitive and Selective Molecular Detection, ACS Nano Letters, 2003; K. Besteman, et. al., Enzyme coated carbon nanotubes as single-molecule biosensors, ACS Nano Letters, 3:6, 727-730; patent applications on carbon nanotubes can be found in Ser. Nos. 11/017,480; 11/144,292; 11/020,024) and electrochemical biosensors rely on charge-based interactions between the protein or tag of interest and the sensor, making each system unreliable in conditions of varying pH and ionic strength. Even a 0.14 M salt solution (similar to human serum) has sufficient Debye screening to shield nanowires from detecting protein binding events. Accordingly, these sensors require the samples to be presented in pure water or precisely controlled salt solutions, an unrealistic requirement for practical settings. For nanowires to detect proteins in serum samples, for example, desalting steps must be performed before detection. Therefore, making the transition from highly sensitive protein detection in an ideal salt solution in the laboratory to diverse biological matrices in the clinical realm has been challenging.

Technologies in magnetic sensor devices typically use ferromagnetic, paramagnetic or superparamagnetic nano- or micron-sized beads to label the detection molecules. In this application, the magnetic beads are “labeled” onto the detection molecules that selectively bind with the biomolecule of interest in a given sample on the sensor directly. The magnetic beads are then placed into the solution where they are applied to a magnetoresistive sensor coated with the capture molecules and allow the attachment of the beads to the sensor. The presence, or absence, of the labeled beads at the magnetoresistive sensor can be measured based upon the magnetoresitive properties of the beads. This method is highly sensitive and can detect low amounts of analytes bound to the magnetic labels. However, current magnetic bead sensing technology has several limitations. Several binding assays have been described that use magnetic particles as labels (U.S. Pat. No. 7,759,134 B2, U.S. patent application Ser. Nos. 11/719,953, 12/094,822, 12/299,775, and 12/299,779).

A device described by D. R. Baselt in U.S. Pat. No. 5,981,297 used an apparatus with a sensor with coating of directly attached binding molecules which binds to the target molecular species. This method is impractical and requires the continued use of new sensors for each diagnostic test.

A device described by M. C. Tondra in U.S. Pat. No. 6,875,621 B2 used a ferromagnetic thin filmed based magnetic field detection system as the sensor.

A device described by B. N. Engel and M. Ward in U.S. Pat. No. 7,172,904 B2 used a detection method using a magnetoresistive random access memory (MRAM) element originally designed for computer harddrive reading heads. This method is prone to short-circuiting due to the unintended use of such devices in a liquid environment.

A device described by H. Fukumoto and M. Nomura in U.S. patent application Ser. No. 10/502,759 used a modified use of a Hall sensor for target molecular species detection.

A device described by V. Fernandez and A. Rida in U.S. patent application Ser. No. 11/214,571 used a capillary chamber containing untethered magnetic or magnetisable microbeads containing both bound and unbounded target molecular species in a uniform magnetic field. It is impossible to separate magnetically bound target analytes from unbounded particles or bounded contaminants which are major drawbacks of this method.

Magnetoresistive sensors such as the Giant Magnetoresistive and Hall sensors are commonly used in computer disk drive magnetic heads and are manufactured by several companies including IBM, NVE Corporation, Philips, Allegro and Honeywell. The magnetic labels are commonly used in molecular biology and are manufactured by several companies including Invitrogen Corporation (Dynal), Thermo Fisher Scientific, Millipore, and Qiagen Corporation.

Current magnetic bead sensing methods suffer from a number of disadvantages
a. Unable to handle samples with different biological matrices
b. Difficulty loading of clinical samples and requires precision liquid handling devices
c. Unable to handle more than one analyte from a single liquid sample
d. Magnetic sensors designed for computer hard drive reader heads and automotive sensing devices that are prone to electrical short circuiting in a liquid environment and requires new sensors to be used for each test
e. Magnetic beads need to diffuse passively towards the detection molecules in a disorganized fashion
f. Open system that is prone to external contamination and not suitable for field analysis
g. Requires handling by trained technicians

### BACKGROUND OF THE INVENTION

### Objects and Advantages

1. To provide a method that can handle different biological matrices for ease of detection.
2. To provide rapid application of clinical samples from sample collection directly for analysis without requiring the use of precision liquid handling devices.
3. To provide a method that can process different and multiple analytes from a single liquid sample that allows for pre-isolation and quick sorting of analytes within 15 to 30 minutes per assay.
4. To provide a surface for the non-direct contact of magnetic sensor with liquid sample that ensures reusability and prevention of short circuit of electronics that drive the sensor.
5. To provide the ability to precisely guide bound magnetic beads towards detection molecules directly adjacent to the magnetic sensors to increase sample detection sensitivity.
6. To provide a closed system that eliminates contamination and presents an economical advantage for use under external field-based applications.
7. To provide resource and space efficient detection methods with minimal waste through the combination of a compact, reusable detection device with a single component, single use disposable cartridge.
8. To provide a method that is simple and rugged that a person without extensive training can operate successfully.
9. To provide a detection method that is quick and provides an accurate, binary diagnosis for each specific analyte.

Further objects and advantages of our invention will become apparent from a consideration of the drawings and ensuing description.

### SUMMARY

A new method and apparatus for detecting the presence, absence, or concentration of one or more target molecular species in a sample suspected of including the target species has been developed using a magnetic field detector. The detector has one or more magnetic field sensors. Closely bound to the sensors are binding molecules which are capable of binding to the target molecules. The sample and one or more species of magnetizable label particles are brought into contact with the detector at the same or at different times. These label particles have attached binding molecules that specifically bind to the target species, the sensor-bound binding molecules, or both the target species and the sensor-bound binding molecules. As a result, label particles bind close to the sensors in numbers proportional to the concentration of the target species. The unbound label particles are removed and the remaining bound label particles are magnetized. The output of a magnetic field detector is monitored to detect the magnetic field produced by the magnetized label particles so as to determine the presence of bound magnetic particles and thereby a positive or negative diagnosis is determined.

The preferred magnetizable label particles are ferromagnetic iron oxide-impregnated polymer beads and the preferred magnetic field sensor is a magnetoresistive material. A magnetic field sensor of a small size can detect the presence or absence of label particles with great sensitivity. The detector can provide a digital binary value to indicate whether or not a label particle is present. The unbound label particles can be removed by applying a magnetic force or electric current to the particles. Examples of the binding molecules are antibodies; poly- or oligo-nucleotides, i.e. DNA or RNA; proteins; synthetic polypeptides; and chelators. Exemplary target molecules include polynucleic acids, proteins, metal ions, and low molecular weight organic species such as toxins, illicit drugs, and explosives.

An especially preferred method for contacting the sample with the sensor is to place the sample in a solution before it is applied to the detection device. Then a second solution containing one or more species of magnetizable label particles is applied to this solution and the combined solution is introduced to a separation apparatus. The magnetizable label particles have attached label-bound binding molecules with a selective binding response to the target molecules located within the specific locations within the separation apparatus. In the presence of target molecules which have been bound to the apparatus, these label-bound binding molecules will sandwich target molecule to form a link between the magnetizable label particle and the separation apparatus.

The apparatus for carrying out the method comprises of a cartridge containing binding molecules capable of undergoing a selective binding interaction with the target molecular species and a housing unit comprising of a magnetic field sensor which is closely positioned to a cartridge when inserted. Magnetizable label particles with binding molecules capable of undergoing a selective binding interaction with the target molecular species are employed so that the label particles can be magnetized by the electromagnets and detected by the magnetic field sensor. Magnetizing means are produced by an electromagnet located within the cartridge directly underneath the target binding molecules. Upon magnetization of the attached label particles by the electromagnet, detection is possible via magnetic field sensors that detect the magnetic field produced by the electromagnets alone and the subsequent field present after the magnetization of the bound magnetized label particles.

The cartridge in which the binding methods are performed comprises of chambers which allows sample flow from a collection vessel to a collection reservoir. The prepared sample solution is injected into an input valve with a small cross-sectional area which pressurizes the liquid sample and forces its flows into and through the branch chambers. At the same time, an electric current is introduced to the cartridge and particles continue to traverse along the length cartridge through electrophoresis. The electrophoretic force drives particle flow through the downstream flow channels, wherein the fluid travels through the channels until they either bind to specific binding sites within the channel or until flow stops at the anode-containing waste collection reservoir.

Sample fluid flow to specific binding locations is supported by the magnetic field generated by the electromagnet located directly beneath the binding site. Target binding molecules are located within each flow channel and magnetically labeled particles selectively bind using a sandwich immunoassay method as they travel through the chamber. Target binding molecules are located directly above a controllable electromagnet which is turned on during this time to increase binding to the target. As sample fluid travels through the downstream flow channel via pressure-driven flow and electrophoretic force, magnetically labeled particles become magnetized once within range of the magnetic field generated by the electromagnet, and orient themselves toward the binding sites within the magnetic field. Non-bound particles are collected at the end of the cartridge in a reservoir through electrophoretic force (and/or a weak magnet located near the end of the cartridge whose magnetic field does not extend far enough to affect the electromagnet of the cartridge).

The cartridge is fully inserted into the detection housing at a downward angle of 45° so that the anode portion of the cartridge lies at a position decline to that the cathode. Electrical connections within the detection housing come into contact with both the anode and cathode and an electric current is introduced to the reaction cartridge. The housing unit comprises a power supply that provides the current which allows the electromagnet in the cartridge to produce an electromagnetic field. A magnetic sensor is located in the housing in the space directly above the electromagnet in the cartridge. The magnetic sensor is positioned normal to the electromagnetic field produced by the electromagnet; when the cartridge is fully inserted into the detection housing, the sensor lies directly above the binding site within the cartridge, opposite the embedded electromagnet. The magnetic sensor is connected to a readout system which indicates the presence of or change within a magnetic field.

The magnetic sensor operates as such. Prior to the introduction of sample fluid, the electromagnet within the cartridge creates a magnetic field upon the complete insertion of the cartridge into the detection housing. The magnetic field is detected and measured by the magnetic sensor located directly above the electromagnet, upon which current flow to the electromagnet is discontinued. As sample solution is introduced and passes through the downstream flow channels, the electromagnet is powered on to create a magnetic field, magnetizing the beads. The magnetized beads orient themselves closer to the binding targets located above the electromagnet. The electromagnet is then powered off and nonspecifically-adhering particles are removed using electrophoretic force and collected in the waste reservoir. The electrophoretic current is then discontinued and once again the electromagnet is powered to magnetize the magnetically-labeled sandwich-bound particles. The magnetic field is once again detected and measured by the magnetic sensor, and the measurement is compared to that of the initial detection. A significant difference in magnetic field strength indicates the presence of binding events.

### DRAWINGS

### List of Reference Numerals

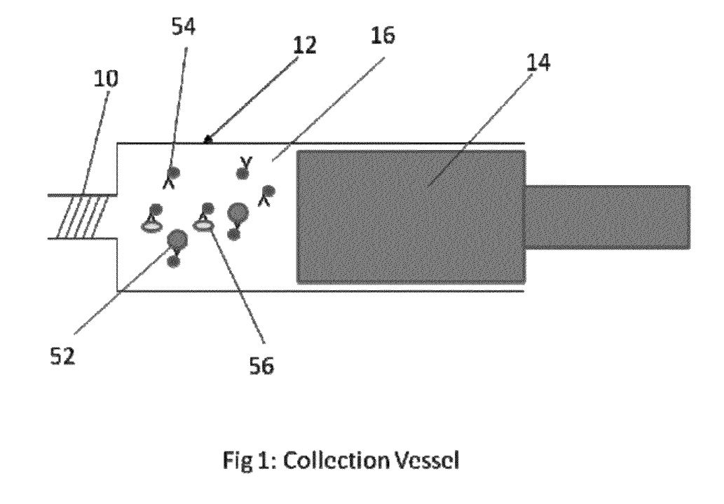

FIG. 1 is a drawing showing the collection vessel containing the specific binding molecules bound magnetic labels where the sample is added

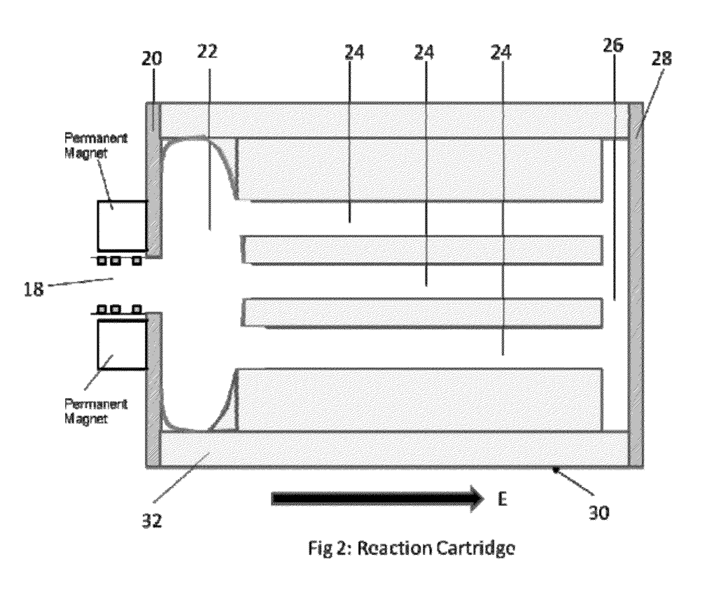

FIG. 2 is a drawing of the assembled reaction cartridge with electrical terminals and plurality of flow channels where bound magnetic labels are detected by immobilized detection molecules

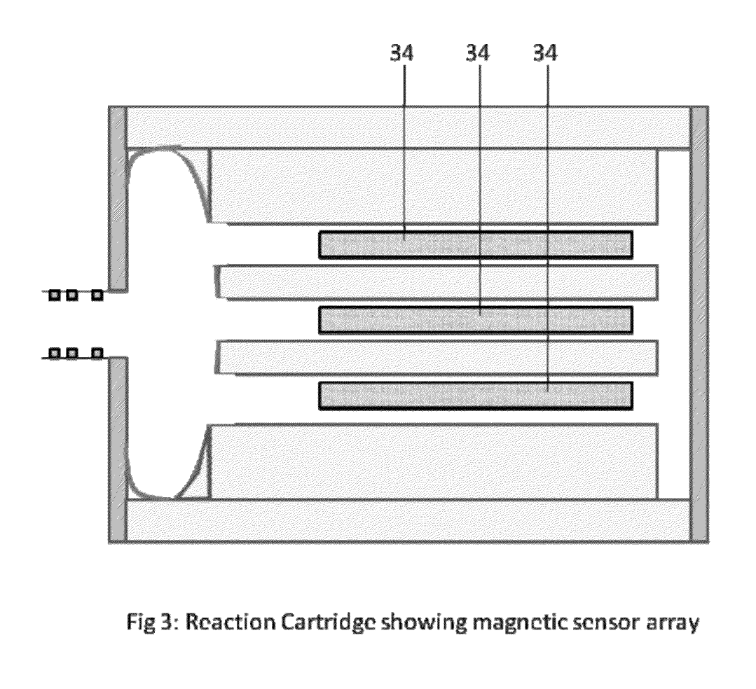

FIG. 3 is a drawing of the reaction cartridge showing a plurality of magnetic sensors array

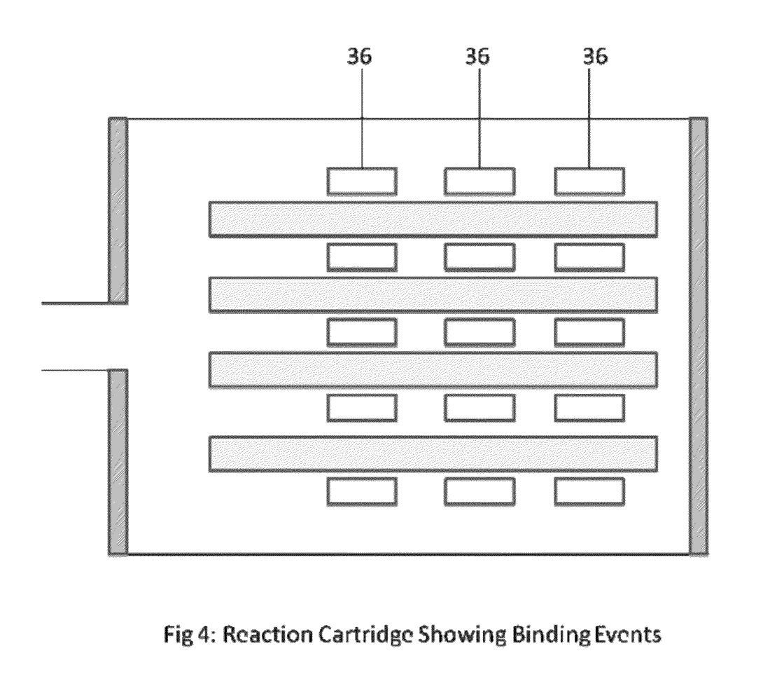

FIG. 4 is a drawing to demonstrate the binding events adjacent to the exterior sensor

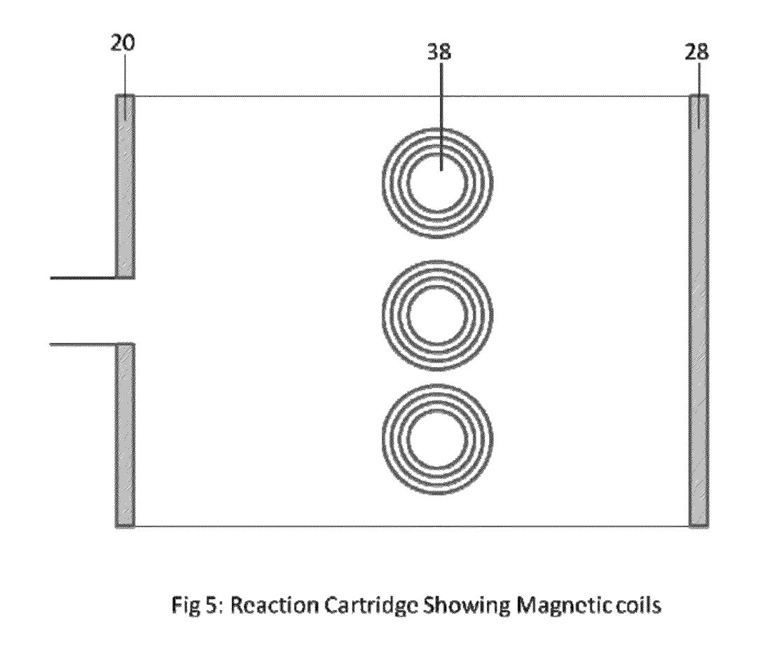

FIG. 5 is a drawing showing the location of magnetic coils on the reaction cartridge

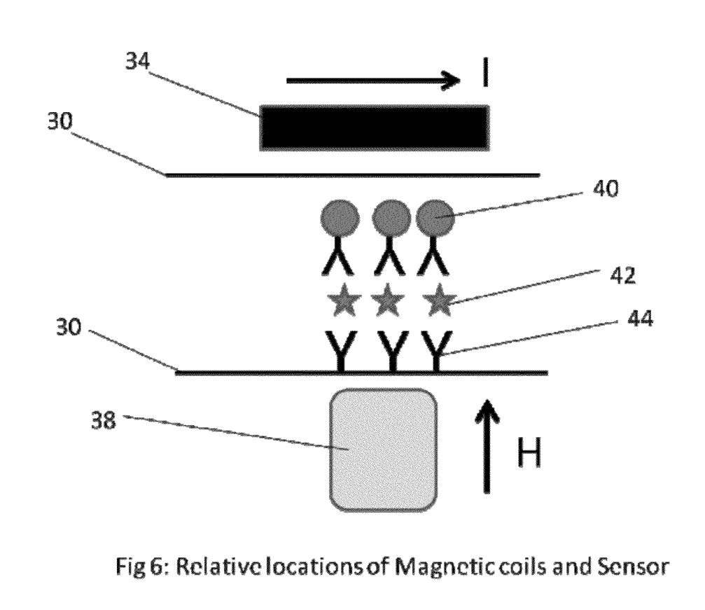

FIG. 6 is a side view of the schematic drawing to show relative locations of the magnetic coils, sensor, and the binding events

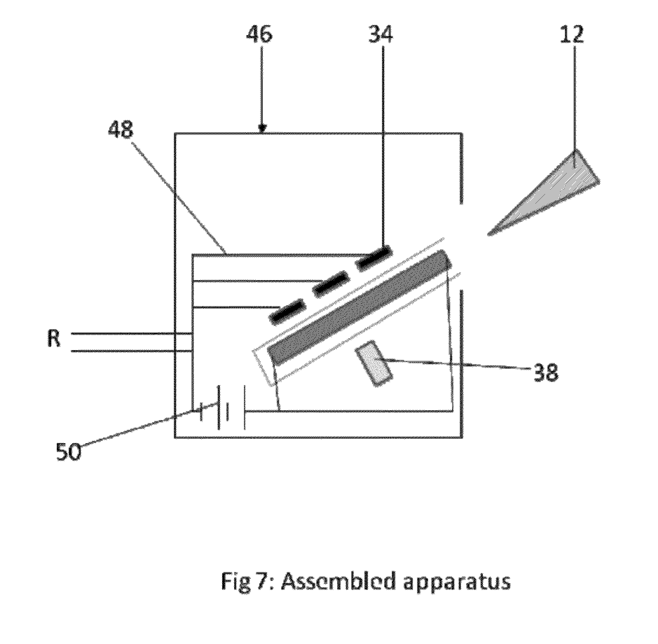

FIG. 7 is a drawing of the assembled apparatus

DRAWINGS-REFERENCE NUMERALS

| | |
|---|---|
| 10 valve | 12 collection vessel |
| 14 rubber-tipped plunger | 16 lysis buffer |
| 18 input valve | 20 cathode terminal |
| 22 distribution chamber | 24 flow channels |
| 26 waste collection reservoir | 28 anode terminal |
| 30 reaction cartridge | 32 non-conducting epoxy |
| 34 magnetic field sensor array | 36 location of binding events |
| 38 electromagnets | 40 magnetic labels |
| 42 target molecular species | 44 antibodies |
| 46 detection housing | 48 electrical connections |
| 50 electrical supply | 52 magnetically bounded target 1 |
| 54 unbound magnetic label | 56 magnetically bounded target 2 |

### DETAILED DESCRIPTION

In FIG. 1 (side view), collection vessel 12 comprised of a receptacle body and a rubber-tipped plunger 14 with a predetermined volume contains a predetermined amount of lysis buffer 16 containing pH7.4 phosphate buffered saline solution and a 5% Tween detergent and a predetermined amount of specific binding molecules bound magnetic labels 80 that can bind directly to target molecular species 52, 54, 56. Up to 200 μl of liquid sample can be added to the collection vessel for processing and subsequently introduced to the reaction cartridge 30 via the valve 10 and input valve 18. Surrounding the input valve is a removable permanent magnet with magnetic strength of at least 500 Guass.

In FIG. 6, the preferred binding assay is a sandwich immunoassay, and the binding molecules are therefore antibodies 44. Antibodies 44, attached to magnetic labels 40 such as ferromagnetic beads of sizes between 0.01 to 4.0 μm, will bind to and immobilize target molecules 52, 54, 56 present in the sample solution.

In FIG. 2, said reaction cartridge 30 for carrying out the method is constructed out of two pieces of glass or plastic material enclosing a distribution chamber 22 that is connected to an input valve 18 and branches out to become two or more downstream flow channels 24. Within each downstream flow channel comprises target binding molecules 36 located directly above the modulatable electromagnets 38 in FIG. 5. The downstream flow channels drain into a waste reservoir 26. The reaction cartridge is prefilled with buffer chemical containing pH7.4 phosphate buffered saline solution and ensures an electrical continuity E within the cartridge. The cartridge is glued together at both ends with conductive epoxy to serve as electrical terminals 20, 28, with the negative cathode terminal 20 in the distribution chamber and the positive anode terminal 28 located in the waste reservoir 26. The lengthwise sides are bound together with non-conducting epoxy 32. These electrical terminals are connected to the electrical supply 50 in FIG. 7. These electrical terminals generate an electrical current E that forces the biological analytes with an innate slight negative charge to move towards the positive anode terminal 28.

The said cartridge 30 has one or more electromagnets 38 in FIG. 5 which are directly bound to the underside of the cartridge 30 and can be modulated during the reaction process. Directly attached within the cartridge above the electromagnets are binding molecules 40, 42, 44 in FIG. 6 capable of undergoing a selective binding interaction with the target molecular species 42. Magnetizable label particles 40 having binding molecules capable of undergoing a selective binding interaction with the target molecular species 42 are used so that said label particles 40 can be influenced by the magnetic fields generated by the electromagnets 38 during the reaction process.

In FIG. 5, said electromagnet 38 is an air-core electromagnet measuring 1 cm in diameter and containing approximately 600 turns of 26 gauge AWG copper wire is bound directly to the bottom of the cartridge, located 1 cm under the detector. A 6 ampere pulse of current is applied to the electromagnet, producing a force of approximately 25 pN on each ferromagnetic particle for 10 ms.

In FIG. 3, said method for detecting the presence or absence of one or more target molecular species 42 in a sample suspected of including the target species 42 has been developed using electromagnets 38 and magnetic field sensor array 34. The magnetic sensor 34 can be a Hall, Giant Magneto Resistance (GMR), or a magnetic sensor. The said sensor array 34 with 48 to 128 individual magnetic sensors in die format which is epoxy glued to a circuit board of preset dimension, and then wire bonded from the pads on the IC to the circuit board. Connections to the circuit board are wired to a detector and the appropriate visualization. Each length of the die is 3301 microns, and width of the die is 367 microns. The center of each bonding pad is 96 microns in from the respective edges of the die. The entire constructed sensor occupies about 60% of the exterior surface adjacent to the reaction cartridge 30.

In FIG. 4, said method for contacting the sample within close proximity to said sensor 34 is to place the sample in a solution which is introduced to said reaction cartridge 30. The solution contains one or more species of magnetizable label particles 52, 54, 56 where the particles have attached label-bound binding molecules 44 with a selective binding response to the target molecules 42. In the presence of target molecules 42 which have been bound to the cartridge 30, these label-bound binding molecules 44 will sandwich target molecule in FIG. 6 to form a link between the magnetizable label particle 40 and the cartridge 30. The sample and one or more species of magnetizable label particles are brought into contact with the adjacent sensor 34 at the same or at different times. These label particles have attached binding molecules that specifically bind to both the target species and the slide-bound binding molecules. As a result, label particles bind close to the sensors 34 and on the adjacent upper surface of the electromagnet 38. The unbound label particles are removed via electrophoretic forces towards the waste collection reservoir 26 and the remaining bound label particles are magnetized 40. The output of a magnetic field detector is monitored to detect the magnetic field produced by the magnetized label particles so as to determine the number of bound magnetic particles and thereby the concentration of the target molecule is determined.

The preferred magnetizable label particles 40 are ferromagnetic iron oxide-impregnated polymer beads (sizes between 0.01 to 4.0 μm) and the preferred magnetic field sensor is a magnetoresistive material. If the magnetic field sensor 34 is of a small size it can detect the presence or absence of label particles with great sensitivity. Examples of the binding molecules are antibodies; poly- or oligo-nucleotides, i.e. DNA or RNA; proteins; synthetic polypeptides; and chelators. Exemplary target molecules include polynucleic acids, proteins, metal ions, and low molecular weight organic species such as toxins, illicit drugs, and explosives.

As used herein, the term “specific binding molecule” refers to a molecule that specifically binds to another molecule through chemical or physical means. Typically, the term “specific binding member” refers to a member of a binding pair such as antigen-antibody binding pairs. Other binding pairs include, but are not intended to be limited to, biotin and avidin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), sugar and boronic acid, and similar molecules having an affinity which permits their association in a binding assay. A binding member may also be made by recombinant techniques or molecular engineering. If the binding member is an immunoreactant it can be, for example, an antibody, antigen, hapten, or complex thereof, and if an antibody is used, it can be a monoclonal or polyclonal antibody, a recombinant protein or antibody, a chimeric antibody, a mixture(s) or fragment(s) thereof, as well as a mixture of an antibody and other binding members. The details of the preparation of such antibodies, peptides and nucleotides and their suitability for use as binding members in a binding assay are well-known to those skilled-in-the-art. A binding member may also be part of a cell, virus or other biological entity that is immobilized on a surface or on a particle.

In FIG. 6, said immobilization of the magnetizable particles 40 with respect to the surface of the device may be by direct binding of specific binding members on the magnetizable particles 40 to specific binding members on the surface of the device, or it may be through intermediaries, such as the analyte molecules 42 themselves or other recognition molecules such as secondary antibodies 44. The common theme is that the presence of the analyte in the test sample can be related to the detection of magnetizable particles 40 that become immobilized on the device by specific binding events. The present invention provides a means to detect whether or not magnetizable particles have become immobilized. By calculating the binding capacities of magnetizable particles to specific target molecules, the concentration of said molecules can be accurately calculated.

### OPERATION OF INVENTION

To use this invention, sample in the form of blood, sweat, semen, or other bodily samples can be used. A pre-determined quantity of sample fluid is added to the collection vessel 12 in FIG. 1 with reaction buffers that dilute and neutralize biochemicals that may interfere with the subsequent binding processes.

In FIG. 7, said reaction cartridge 30 is first fully inserted into the detection housing 46 at a downward angle of 45° so that the anode 28 portion lies at a position decline to that the cathode 20. Electrical connections within the detection housing 46 come into contact with both the anode and cathode and an electric current I is introduced to the reaction cartridge 30. The north and south terminals H of the reaction cartridge's electromagnet also come into contact with the electrical connections 48 of the detection housing 46, allowing for a current I to be applied selectively to the electromagnet 38 as well. Located within the detection housing 46 is the detection sensor 34. When the cartridge is fully inserted into the detection housing, the sensor lies directly above the binding site within the cartridge, opposite the embedded electromagnet 38.

In FIG. 2, sample is introduced to said reaction cartridge 30 through input valve 18 with small cross sectional area relative to the distribution chamber 22. Said removable permanent magnet is added to allow bound magnetic labels to concentrate around the input valve. After the application of the sample into the cartridge 30, the permanent magnets are removed. The increased pressured through the valve 18 forces the sample fluid to flow from the valve into the flow channel 24 and ultimately through the downstream flow channels for binding events 36. As the pressurized sample fluid enters the reaction cartridge 30, an electric current I and a magnetic field H are introduced to the reaction cartridge 30 in FIG. 6. Sample fluid is further driven through the downstream flow channels via the electrophoretic force, wherein the fluid continues to traverse through the channels until they either bind to specific binding sites within the channel or until flow stops at the anode-containing waste reservoir 26. Sample fluid flow to specific binding locations is supported by the magnetic field H generated by the electromagnet 38 located directly beneath the binding site 36. As sample fluid travels through the downstream flow channel 24 via pressure-driven flow and electrophoretic force, once magnetizable label particles 40 are within range of the magnetic field generated by the electromagnet 38, they become magnetized and orient themselves toward the binding sites within the magnetic field H.

In FIG. 6, electric current I is applied upon the introduction of the sample solution. After a specified time delay from the application of the electric current and followed by the discontinuation of the said current by electronic controller R, a voltage is applied to the electromagnet, creating a magnetic field H. The magnetic field H magnetizes the magnetizable label particles 40 within the sample solution as they flow within range of the electromagnet's magnetic field H. The magnetization of the labeled particles increases binding events as magnetized labeled particles are drawn closer to binding sites via magnetic force.

In FIG. 3, the sensor 34 operates as follows. Prior to the introduction of sample fluid, the electromagnet 38 within the cartridge creates a magnetic field H upon the complete insertion of the cartridge 30 into the detection housing 46. The magnetic field H is detected and measured by the magnetic sensor 34 located directly above the electromagnet 38, upon which current flow to the electromagnet 38 is discontinued. As sample solution passes through the downstream flow channels, an electromagnet creates a magnetic field H that magnetizes the beads 40. The magnetized beads 40 orient themselves closer to the binding targets located above the electromagnet. Before the sensor 34 is activated, the electromagnet is powered off and nonspecifically-adhering particles 54 are removed using electrophoretic force E and collected in the waste reservoir 26. The electrophoretic current is then discontinued. Before the detector 34 is activated, an electromagnet 38 is used to magnetize the magnetically-labeled sandwich-bound particles in FIG. 6. The magnetic fields H required for this purpose are large, and, to avoid the requirement of correspondingly large and heavy instrumentation, are provided by sending a brief (10-100 ms) pulse of current through an air-core electromagnet coil 38. The magnetic field is detected and measured by the sensor 34, and the measurement is compared to that of the initial detection. A significant difference in magnetic field strength indicates the presence of binding events.

Furthermore, the binding assay used in the present invention is not limited to antibody-based detection. Any selective binding agent can attach the beads to the cartridge-bound binding molecules. Again the amount of label particles that ultimately remain bound will be a function of the concentration of the target molecules.

In FIG. 4, as the magnetizable label particles 52, 54, 56 traverse the length of the cartridge 30 via an electrical current E, the bound analytes 52, 56 are sorted according to their relative sizes and innate charge. The electromagnets 38 are turned on to magnetize the particles and draw them closer to the binding sites located directly above the electromagnets 38. The magnetizing means provided for the magnetization of attached label particles and the detection means both rely on the production of a magnetic field H produced by the electromagnets 38 and the subsequent employment of the magnetic sensor 34 to detect the difference in magnetic field H produced by the electromagnets 38 alone and once again in the presence of attached magnetized label particles 40. A difference in magnetic field detected by the magnetic sensor indicates the presence of target molecule and thus a positive result is determined.

In FIG. 7, said sensor 34 can provide a digital binary value R to indicate whether or not a label particle is present.

It is understood that the foregoing detailed description is given merely by way of illustration and that many variations may be made therein without departing from the spirit of this invention.

### CONCLUSION, RAMIFICATIONS, SCOPE OF INVENTION

Thus the reader will see that the invention Electromagnetic Multiplex Assay Biosensor provides a sensitive, flexible in operation, yet economical device that can be used by persons with low technical expertise and training. Our invention presents a method that can be applied to a variety of uses in molecular biology, clinical diagnostics and biodefense by overcoming signal distortion that occurs in various biological sample matrices due to differences in ionic strength, pH, temperature, and autofluorescence.

While our above description contains many specificities, these should not be construed as limitations on the scope of the invention, but rather as an exemplification of one preferred embodiment thereof.

Accordingly, the scope of the invention should be determined not by the embodiment (s) illustrated, but by the appended claims and their legal equivalents.

What is claimed is:

1. A method of multiple detection of different target molecular species in a liquid sample using a magnetic biosensor device comprising
a. a collection vessel configured to receiving a sample and comprises multiple binding elements that bind said target species with magnetic labels;
b. an input valve configured for introducing the liquid sample into a reaction cartridge through a hole;
c. the said reaction cartridge with electrical terminals that provide an directional electrical interconnection;
d. a distribution chamber configured to receiving the bound targeted species through said hole from a input valve;
e. a plurality of branch flow channels wherein each branch flow channel is in fluid communication with the main flow channel and the reservoir and comprises detection regions containing the binding elements;
f. a electric field passing through said flow channels providing differential mobility of labeled target species;
g. a magnetic field generator configured for generating both controllable electromagnetic fields wherein alteration of the binding elements via specific binding to the target species produce a resonance response of the magnetic labels bound target species;
h. detecting said magnetic labels with bounded molecular species with a plurality of predetermined number of magnetic sensors linked together in a parallel configuration means to detect target molecular species rapidly, specifically and reliably.

2. Method according to claim 1 wherein detection of said different target molecular species is determined and wherein the detection binding molecular layer is immobilized on the surface of a branch flow channel in a position adjacent to the magnetic sensor.

3. Method according to claim 1 wherein the detection binding molecular layer binds directly to the target molecular species.

4. Method according to claim 1 wherein the composition and location of detection binding molecular layers in a plurality of flow channel is different and a specific said layer recognizes a specific said target species.

5. Method according to claim 1 wherein labeled target particles flow and bind to detection regions in branch flow channels through a magnetic field generated by controllable electromagnets and directional electrical field.

6. Method according to claim 1 wherein magnetic fields are generated by controllable electromagnets located within the cartridge.

7. Method according to claim 1 wherein unbound particles are eluted from the branch flow channels into the reservoir through a directional electrical field.

8. Method in claim 1 wherein target molecular species can be tumor cell, mammalian cell, plant cell, virus particle, bacterium cell, deoxyribonucleic acid fragment, ribonucleic acid fragment, protein, oligonucleotide, peptide, antibody, prion, and environmental contaminant.

9. Method of analyzing multiple detection of target molecular species using a magnetic sensor device wherein the detection of different said species is determined, comprising the steps of
a. Binding of said targets to magnetic labels;
b. Bound magnetically labeled targets move electrophoretically in directional electrical field in flow channels means to provide differential mobility of labeled target species;
c. Said species separate according to size and charge;
d. Contacting the detection binding molecular layer and the magnetically labeled targets;
e. Detecting the magnetic label with plurality of magnetic sensors.

10. Device for analyzing the presence of multiple different target molecular species within a self-contained reaction cartridge.

11. Device for according to claim 5 & 6 wherein the electromagnets used in the generation of magnetic fields is an electromagnet, solenoid or air core inductor.

12. Device for according to claim 7 wherein the detection sensor used in the detection of binding is a Hall sensor, Giant Magneto Resistive sensor or Inductor coil.

13. Device for according to claim 7 wherein the detection sensor used in the detection of binding is housed in a unit wherein the sensor is located directly above the electromagnets so that the magnetic field produced is normal to the sensor.

14. Device for according to claim 7 wherein the detection binding molecular layers are immobilized on a surface independent of the adjacently located magnetic sensor.
Device for according to claim 7 wherein the detection sensor can provide real time measurements of magnetic fields generated within or by the reaction cartridge means to allow highly selective and sensitive detection of target molecular species in the detection of important diseases.

15. A kit of parts for detection of target molecular species, comprising a device according to claim 7 in combination with a suitable molecular species detection system and/or a binding composition.
